(19) United States
(12) Patent Application Publication
(10) Pub. No.: US 2011/0306921 A1
(43) Pub. Date: Dec. 15, 2011

(54) THERAPEUTIC DEVICE AND METHODS OF USING AND MAKING SAME FOR MULTIMODAL STIMULATION OF LIVING TISSUE

(75) Inventor: Evelene Huguenin Hawley, Florence, MA (US)

(73) Assignee: PROTOPEUTICS, INCORPORATED, Easthampton, MA (US)

(21) Appl. No.: 13/157,156

(22) Filed: Jun. 9, 2011

Related U.S. Application Data
(60) Provisional application No. 61/353,334, filed on Jun. 10, 2010.

(51) Int. Cl. A61N 1/04; B32B 38/08; B32B 38/00; A61N 1/30

(52) U.S. Cl. 604/20; 607/152; 156/229; 156/244.24

(57) ABSTRACT

Therapeutic devices for stimulation of living tissue are provided. Multimodal stimulation may be mechanical, electrical, chemical, sensory and combinations thereof. The devices have a matrix sufficiently elastic so that when attached to the tissue, the matrix is capable of applying force upon the tissue and compliantly responding to force resultant from motion of the tissue. The devices also have an electrically conductive region contained within the matrix, so that, selectable locations of the device are capable of being in electrical communication with a power source and with the tissue. Methods of stimulating tissue as well as methods of making the devices are also provided.

### CROSS-REFERENCE TO RELATED APPLICATION(S)

This application claims priority from U.S. Provisional Patent Application Ser. No. 61/353,334, filed Jun. 10, 2010, which is incorporated herein by reference.

### TECHNICAL FIELD

The present invention relates to platforms or tapes for and the resultant multimodal therapeutic devices derived therefrom as well as methods of using and making such devices.

### BACKGROUND ART

Development of devices, platforms and garments to comfortably and effectively provide mechanical stability to portions of the anatomy has advanced over time. Also, a plethora of electrostimulation techniques: square wave transcutaneous electro nerve stimulation (TENS), biphasic neuromotor electrical nerve stimulation (NMES), interferential stimulation, high voltage pulsed galvanic stimulation, Russian current, low intensity stimulation (LIS), and iontophoresis have been shown to be beneficial in providing strengthening, pain management or amelioration of physical and psychological maladies. Customary application of these signals is applied through a pair of monopolar patch electrodes. One patch is attached to the cathodic source and placed at one end of the body of the muscle and the other is attached to the anodic and placed at the opposite end of the muscle. Research being conducted to support the development of implantable electrodes is evidencing that maximum contraction and torque can be achieved through exciting specific motor trigger points for each muscle. The motor trigger points for rats have already been thoroughly mapped in the forelimbs (Kanchiku et al, “Neuromuscular Electrostimulation Induced Forelimb Movement in a Rodent Model,” Journal of Neuroscience Methods, 167 (2008) p.317-326) and hindlimbs (Jung et al, “Chronic Neuromuscular Electrostimulation of Paralyzed Hindlimbs in a Rodent Model,” Journal of Neuroscience Methods, 183 (2009) p.241-254). Additionally, this research demonstrates that localized excitation requires significantly less current. In a clinical setting, lower current should translate to less pain and irritation, higher patient acceptance, and products more easily adapted to pediatric service and other underserved populations.

In addition to the aforementioned exteroceptive (the senses/mechanics) and interoceptive (for example, pain perception) therapeutic treatments, many advances have also occurred in the area of sensory integration treatments. Kinaesthesia involves the ability to sense whether/how motion of the body is occurring. Proprioception is the awareness of limb location. Although clinicians refer to these as separate sense, researchers note that they share some common receptors of origin. (Proske et al, “The Kinaesthetic Senses”, Journal of Physiology, 587, (2009) p.4139-4146) Muscle spindles generate information on motion as well as position. Muscle spindles need to be activated in groups in order to be interpreted by the brain while joint afferents, located in ligaments, can inform the brain of joint dislocation by the stimulation of one receptor. (Macefield et al, “Perceptual Responses to Microstimulation of Single Afferent Innervating Joints, Muscles, and Skin of the Human Hand,” Journal of Physiology, 429 (1990), p.113-129) Ruffini endings in cutaneous tissues are stretch receptors which supplement the aforementioned receptors. They collectively relay information to the brain which it then interprets as effort, external force, position, etc.

The integration of multiple sensory inputs is how the brain resolves peripheral sensory ambiguities. There would be utility in developing therapeutic devices which would provide heightened multimodal stimulus to patients in all of these areas.

### SUMMARY OF THE INVENTION

A therapeutic device for stimulation of living tissue is provided in a first embodiment. The stimulation may be mechanical, electrical, chemical, sensory and combinations thereof. The device has a matrix capable of attaching to living tissue, the matrix being sufficiently elastic so that when attached to the tissue, the matrix is capable of applying force upon the tissue and compliantly responding to force resultant from motion of the tissue. The device also has an electrically conductive region contained within the matrix, so that, selectable locations of the device are capable of being in electrical communication with a power source and with the tissue. The device may also have an electrode in electrical communication with the tissue. The conductive region may include a fluid solution (<5,000 centipoise), a viscous medium (>5,000 centipoise), metal foil, electroplated metal, or a composite of these. The device may also have an outer adhesive layer bonded to the matrix. In further embodiments, flow of the fluid solution or viscous conductive medium may be promoted in response to force exerted upon the matrix. In an additional embodiment, electrical communication is maintained when the device has been strained to 225%. Other device embodiments may be capable of, in response to device elongation and contraction caused by tissue motion, facilitating measurement of motion of a limb, the limb proximal to the tissue and/or facilitating measurement of an angle exhibited by a joint, the joint proximal to the tissue. Further embodiments may have a layer of pharmaceutical material, such as analgesic, anesthetic, anti-inflammatory and vitamin capable of being delivered to the tissue via electromotive drug administration.

Additional embodiments provide methods for stimulating living tissue of a subject. Methods include selecting stress, electrical triggering and sensory subject locations, providing the therapeutic disclosed above, mapping the device by spatially matching the subject locations with the selectable locations of the device, attaching the mapped device to the subject; and powering the device.

Further embodiments provide methods of making the therapeutic device. For devices that include electroplated metal as the conductive region, a provided method of making includes: extruding thermoplastic material on carrier material to make first and second plies, creating a mask, applying tension to the second ply, affixing the mask to the tensioned second ply, depositing conductive material on masked second ply, applying conductive gel upon deposited conductive material, removing mask from the second ply, releasing tension from the second ply, laying up the first ply with the second ply, and sealing the plies together. For devices that include fluid or viscous conductive media, a provided method includes: extruding thermoplastic material on carrier material to make first and second plies, sealing the first to the second ply leaving predetermined wells between the plies and injecting the conductive medium into the wells. The method may also include the step of providing adequate activation energy to crosslink the plies.

### BRIEF DESCRIPTION OF THE DRAWINGS

FIGS. 1a-1h illustrate an exemplary clinical case study for which multimodal treatment may be beneficial in accordance with an embodiment. FIG. 1a introduces the case depicting the desired adjustment; FIG. 1b shows placement of a device embodiment upon the body of the subject; FIG. 1c depicts [left-most]: appropriate tensioning, muscles to be stimulated and how the locations where and how a practitioner would activate; [center]: whole muscle treatment using the device in monopolar mode, and [right-most]: target centered muscle treatment using the device in bipolar mode. FIGS. 1d-1h depict of in-situ placement and operation of exemplary device embodiments.

FIGS. 2a-2b depict a monopolar therapeutic device in accordance with an embodiment.

FIG. 2c depicts a perspective view of a bipolar configuration.

FIG. 3a depicts front view of encapsulated buses/nodes;

FIG. 3b depicts front view of encapsulated buses/nodes in tension;

FIG. 3c depicts a detailed internal flow profile of an encapsulated bus in tension;

FIG. 3d depicts a front view of another encapsulated buses/nodes with flow features;

FIG. 3e depicts a front view of another encapsulated buses and nodes with flow features in tension; FIG. 3f depicts a detailed internal flow profile of another encapsulated bus with flow features in tension;

FIG. 3g depicts the longitudinal elongation of the bus in tension and the increase in the included angle of the arc;

FIG. 3h depicts a profile view showing uneven expansion of encapsulating channel away from weld midline;

FIG. 3i depicts the internal pressure variations in the encapsulated channel; and

FIG. 3j depicts fluid displacement with regard to dynamic motion of a joint, all in accordance with exemplary embodiments.

FIG. 4 depicts a perspective view of the insertion site, appliance, and tape interface in accordance with an embodiment.

FIG. 5 is a manufacturing flow chart for encapsulating fluid or viscous conductor within insulative thermoplastic films in accordance with an embodiment.

FIG. 6 is a manufacturing flow chart for depositing a conductive metal on thermoplastic sheets in accordance with an embodiment.

FIG. 7 is a manufacturing flow chart for encapsulating a conductive fluid with thermoset plies in accordance with an embodiment.

FIG. 8a depicts a plan view of orthogonal nodes and buses with flow enhancing features.

FIG. 8b depicts a detail view of orthogonal nodes and buses with flow enhancing features and with ventilating pores.

FIG. 8c depicts a cross-sectional view depicting welds and connectivity with an appliance, all in accordance with another embodiment.

FIG. 9 depicts, in accordance with a further embodiment, a zigzag bus and node configuration directed toward bipolar applications.

### DETAILED DESCRIPTION OF EXEMPLARY EMBODIMENTS

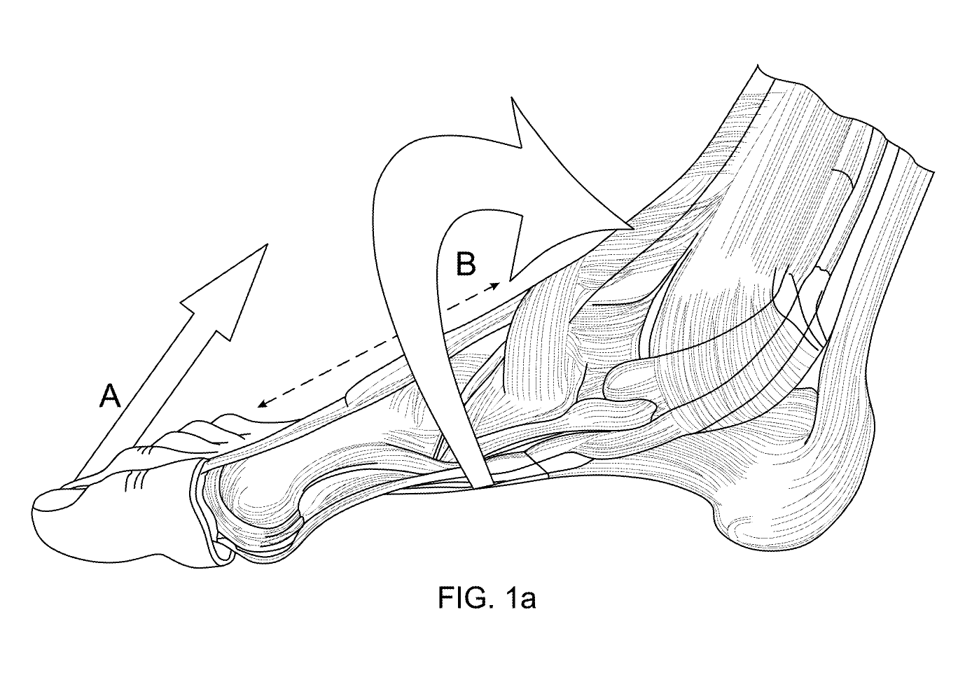

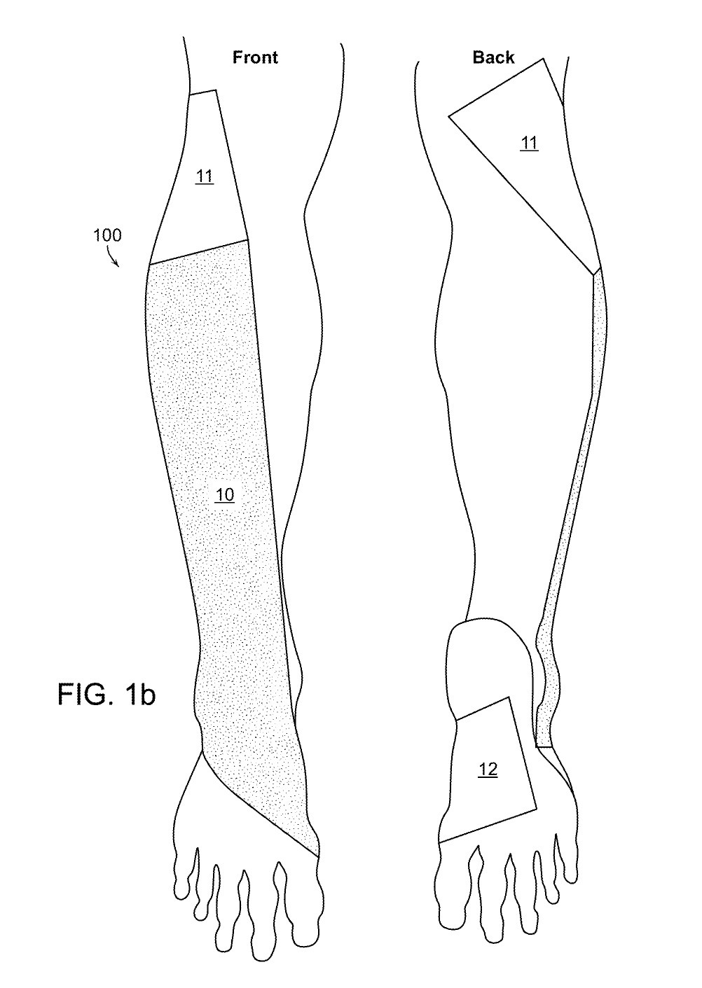

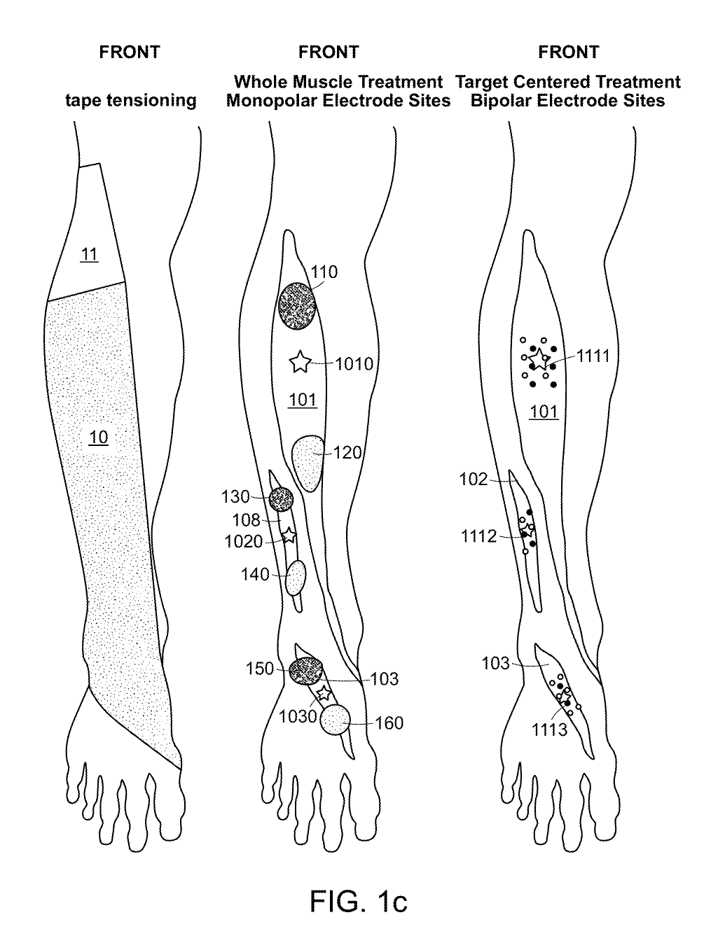

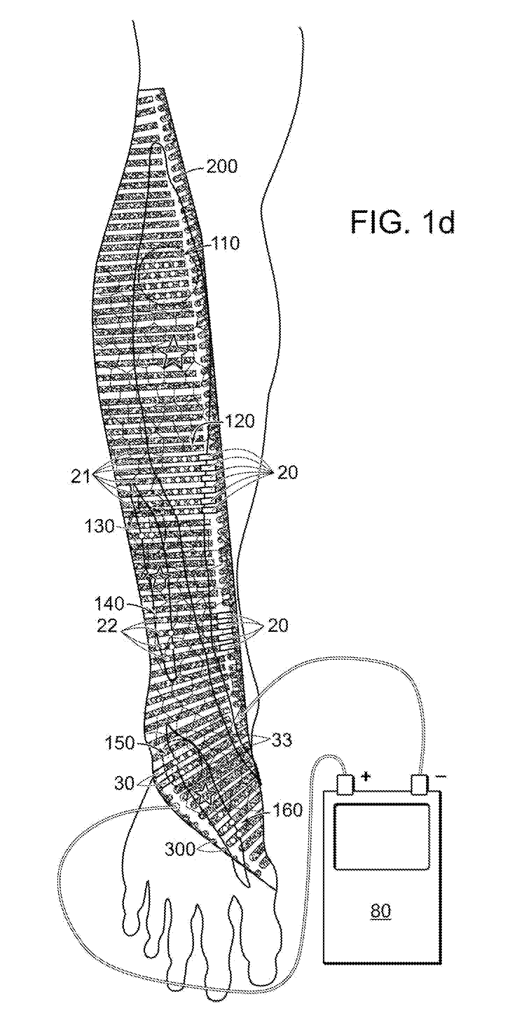

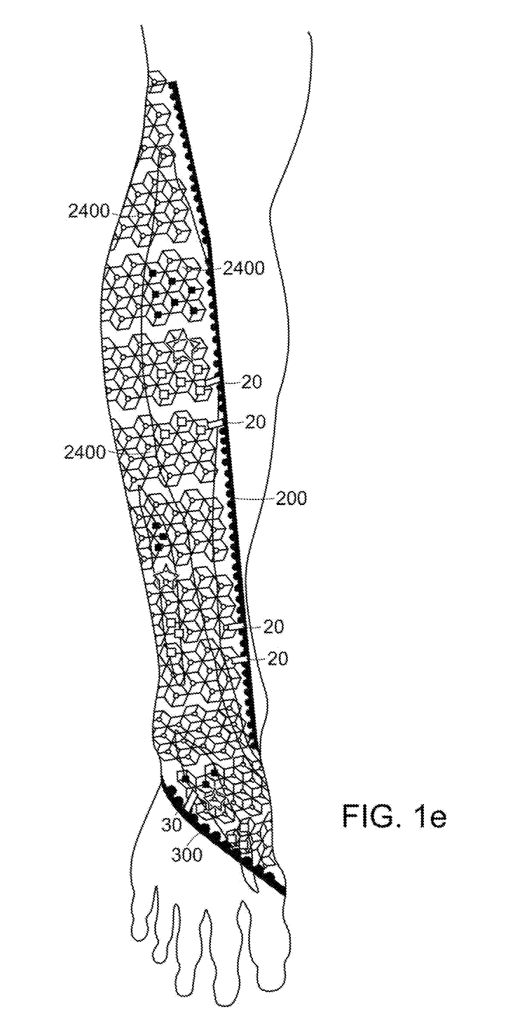

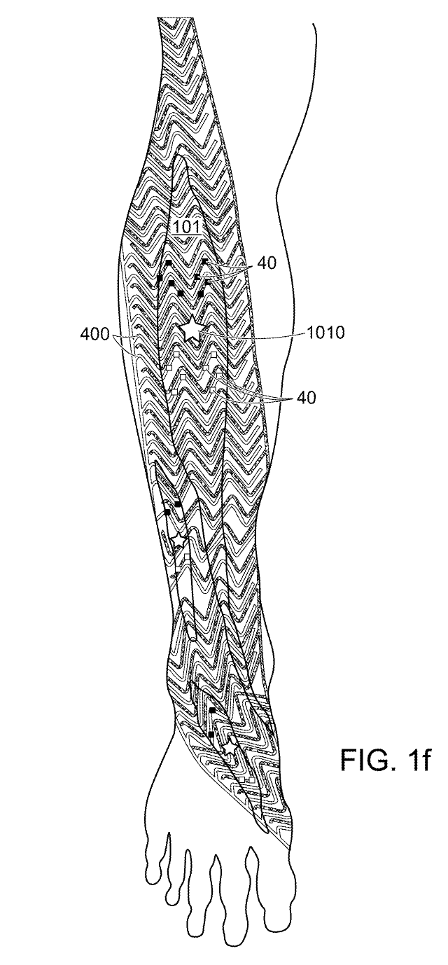

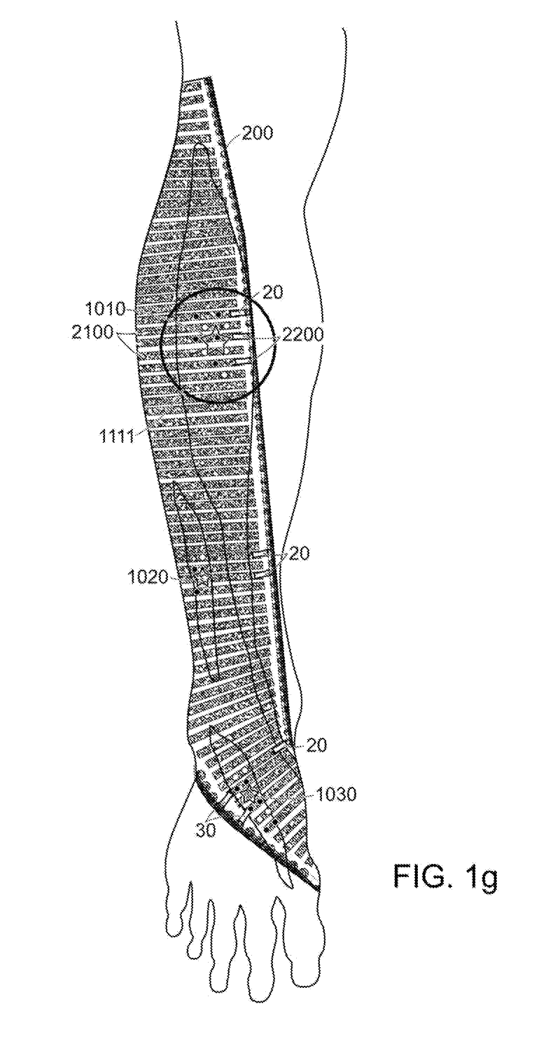

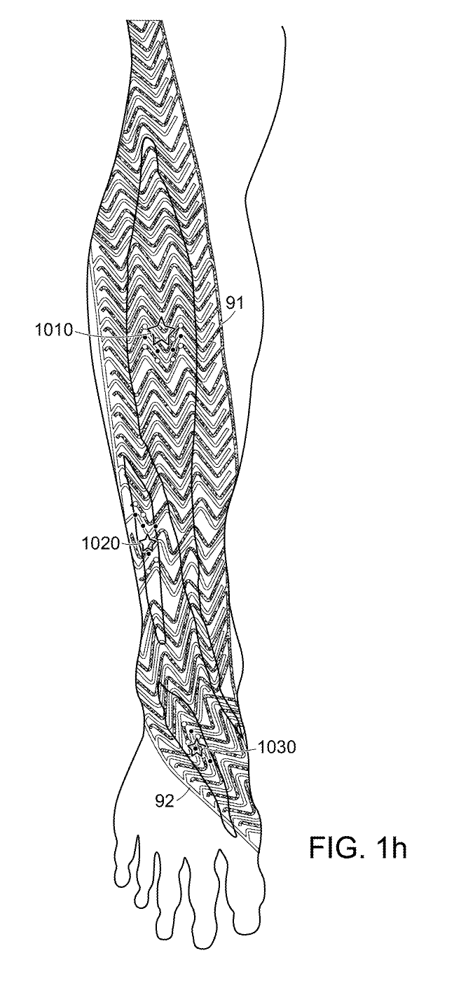

FIGS. 1a-1c serve to illustrate an exemplary clinical case study for which multimodal treatment may be beneficial in accordance with an embodiment. This exemplary study, while involving only leg/foot muscles and anatomy, is not to be construed as limiting device and method embodiments described herein to only those portions of the anatomy. A medical professional has determined that a patient will benefit from both increased dorsiflexion of and reduced pronation of the right foot. As shown in FIG. 1a, what is mechanically desired is for tension to be applied in direction A to raise the toes, thereby decreasing the angle presented between the shin and the foot. Added tension is also required to rotate the foot about axis B. Traditionally with such a diagnosis, the required stresses to perform such adjustments would be applied through restrictive bracing, wrapping the region with tape and/or the attachment of some form of appliance to the area.

A multimodal approach may achieve superior long-term results. In addition to the mechanical treatment, muscle stimulation to achieve strengthening of three particular muscles (denoted 101, 102, and 103 in FIG. 1c) may lead to sustainably improved foot dorsiflexion and alignment. Strengthening of these muscles will occur with use of tailorable electrical signal input. Furthermore, this multimodal approach may heighten proprioceptive input in the joints of interest through the application of tension, thereby enhancing the possibility for long term treatment success. Tension adds sensitization to the skin and to joint receptors and is beneficially applicable in calf and foot region 10. Merely adherent attachment (little to no stress or strain) of multimodal device 100 in regions 11 and 12 may be optimal as illustrated in FIGS. 1a and 1b. Unlike braces which support without any rehabilitative capabilities, the multimodal stimulation of continuous kineasthetic and proprioceptive stimuli coupled with periodic electrical nerve training is intended to strengthen the patient and build self-regulating body awareness.

Device 100 has a matrix capable of attaching to the skin in ways known to one of skill. The matrix is sufficiently elastic so that when attached to the skin as exemplified in the FIG. 1 case study, device 100 is capable of applying force upon the leg and foot and compliantly responding to force resultant from motion of the leg/foot. In the illustrated embodiments, device 100 has contained within the matrix an electrically conductive region so that, when attached, selectable locations of device 100 are capable of being both in electrical communication with a power source and in selective physical and selective electrical communication with the attached skin and, therefore, with the neighboring muscle, nerve and/or ligament tissue.

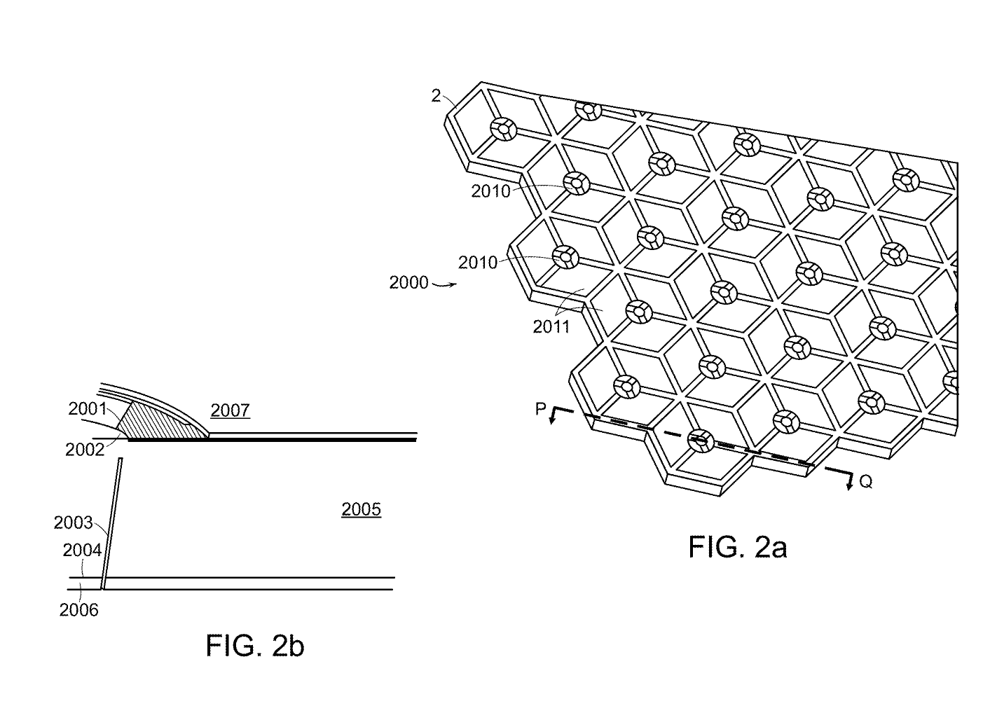

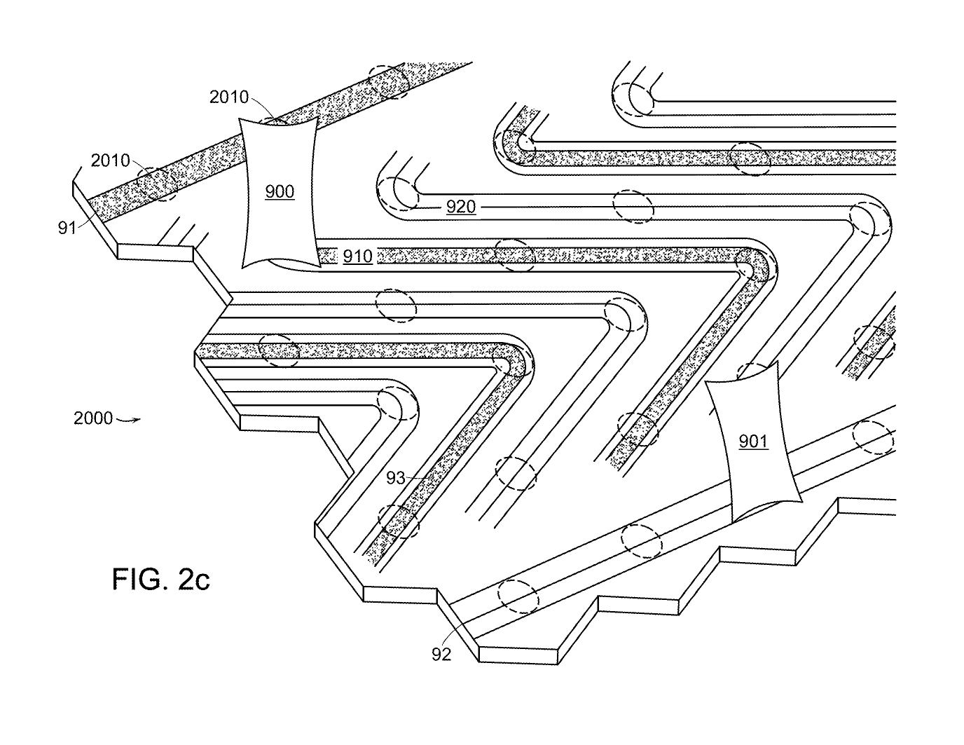

FIGS. 2a-2b feature an embodiment of a composite tape/platform 2000 that forms the underlying structure for fabrication of an embodiment of a multimodal therapeutic device 100. FIG. 2a depicts an initially flat composite tape 2000 which is sufficiently compliant and elastic to be shaped and juxtaposed upon living tissue without deleterious effect to intended signal generation and signal maintenance over time. In this embodiment, tape 2000 is here comprised of contiguous hexagonal cells 2011. The size and shape (shape is not to be construed herein as limited to being hexagonal) of cells 2011 will depend upon factors including but not limited to electrical parameters required for desired therapeutic effect and the size, shape and nature of the living tissue to be stimulated. Cells 2011 are contiguous; however, there may be, in other embodiments, regions within the matrix of a produced tape 2000 devoid of any cells. Refer, for example, to FIG. 1 e and the pertinent discussion below.

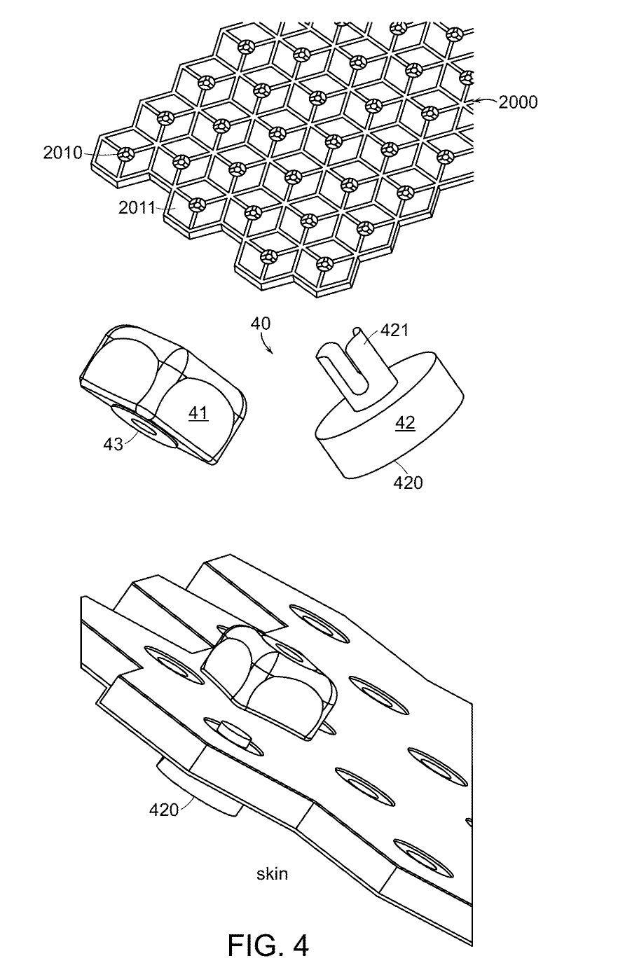

Centrally disposed in each cell 2011 shown in FIG. 2a is an insertion point 2010; insertion points 2010 are die cut or otherwise provided through the thickness of tape 2000 forming sites on the tape through which electrodes are to be selectively placed by a practitioner. The term electrode is meant to include both active electrodes (e.g. TENS) and passive electrodes (e.g. EMG). As treatment plans evolve, the practitioner may be able to add electrode to different sites 2010 and, perhaps with care, remove electrodes from other sites 2010 as desired. Referring to FIG. 4, an embodiment of a two-piece appliance 40 is illustrated. For this particular electrode 40 embodiment, first portion 41 is keyed, threaded, press fit, snap-fit into, or otherwise rigidly attachable to through post 421 of second portion 42 following placement of second portion 42 into a selected insertion point/electrode site 2010. In this embodiment, contact element 420 of second portion 42 comprises the functional elements of a transcutaneous electrode, and is designed to contact the tissue. Conductive surface 43 of first portion 41 is designed to electrically couple with conductive surface 2001 (FIG. 2) of the matrix and via through post 421. Locating the conductive junction on the upper surface (away from the skin) of the tape 2000 is intended to minimize the possibility of unintentional arcing to skin. The conductive pattern shown in FIGS. 2(a)-(b) and in FIG. 4 is a monopolar embodiment. Again referring to FIGS. 2a-2b, due to the shared contiguous sides 2 in this embodiment, if two or more electrical elements are placed into matrix 2000 via insertion points 2010 then electrical communication exists between those elements. If any single hexagonal cell 2011 is connected to a power source (not shown) then all inserted electrodes in the contiguous region will be active and will function as a monopolar electrode array.

Referring to FIG. 2b, platform 2000, viewed cross-sectionally through direction PQ, is composed of sequential layers including top electrically insulative surface 2007, conductor 2001, an optional conducting gel layer 2002, compliant body 2005, bottom electrically insulative layer 2004 and optional adhesive layer 2006, if needed for attachment of tape 2000 to the living tissue to be stimulated. Feature 2003 is a die-cut partial-cut/perforation which is not required of all embodiments. Adhesive layer 2006 may not be necessary in other platform embodiments if either the top or bottom layer (2007 or 2004), respectively, is sufficiently adherent to itself and tape 2000 is thus capable of being securely wrapped about itself and the living tissue. As a practical matter, tape 2000 of device 100 would also have a barrier layer (not shown) to be stripped off of adhesive layer 2006 prior to use/activation. The body of platform 2000 is formed from essentially insulative matrix materials (2004, 2007, 2005) that must be sufficiently compliant and elastic in nature. For example, it may be formed from some initially viscous thermoplastic elastomer, from an elastomer film, from a vulcanizing rubber, such as isoprene or neoprene, or from a silicone. Examples of thermoplastic materials include, but are in no way limited to, polyurethane, styrene-butadiene alloys, polyether-ester elastomers, polyamide elastomers, and polyolefin blends (e.g. Santaprene®, Dynaflex®, Versaflex®, Kraton®, Grilamid®). Sealing properties of tape 2000 (particularly proximal to insertion points 2010) must be such that inserted electrodes 40 and the conductive material 2001 (and or 2002) contained within tape 2000 with which electrodes 40 make electrical contact to complete stimulation circuitry, remain secure during anticipated stresses and strains occurring during device operation.

For some proposed applications of therapeutic device 100, an essentially continuous conductor 2001 and “continuous” optional conducting gel layer 2002 may be useful. With reference to FIG. 2, layer 2001 is continuous in that the complete contiguous pattern, with shared contiguous sides 2 of cells 2011 (or some fraction of sides 2 thereof) is connectable to a single pole of the attachable power source. However, the particular nature of the desired electrical stimulation may make the utilization of a discontinuous conducting layer, which has the capability of having opposite polarity electrode sites in closer proximity to one another, more advantageous. Referring to an embodiment depicted in FIG. 9, conducting layer 90 is discontinuous. Nodes 93 are shown to provide conductive pathways to either first bus 91 or second bus 92. As first bus 91 may be cathodic with respect to the power source and second bus 92 may be anodic, this electrical connection will result in particular adjacent nodes 93 having opposing polarities. FIG. 9 illustrates a zigzag node arrangement. As described below, nodes 93 may alternatively be arranged essentially orthogonal to the alignment of the buses 91 and 92 (or may be disposed in any other geometric arrangement that proves to have utility based upon the desired location and desired polarity of associated stimulation sites). FIG. 9 illustrates nodes 93 that are permanently in contact with one particular bus 91 or 92; however, such contact may (in further embodiments) be selectively effected and removed through use of jumper connectors creating further flexibility in the electrical configuration of platform 2000. Refer to FIG. 2(c). In this platform 2000, nodes 93 and buses 91 and 92 are illustrated wherein nodes and buses are not permanently connected. Potential insertion points 2010, are shown as dashed areas as they made be punched through platform 2000 as desired. Clearly, indexing of potential insertion points 2010 with conductive node locations is essential. For example, if node 910 were placed in electrical communication with bus 91 using jumper 900 and node 920 were placed in electrical communication with bus 92 using jumper 901 (and if bus 91 is connected anodically while bus 92 is cathodically connected) adjacent anodic and cathodic electrode sites are capable of being created by selectively punching or otherwise inserting electrodes through the thickness of platform 2000. The exemplary embodiment of FIGS. 2(c) and 9 depict uniform spacing of nodes/potential electrode sites/insertion points 2010. The zigzag or wave pattern is convenient so as to reference the location and relative spacing of the electrodes. By placing 1) electrodes 40 at the extrema of the waves on the nodes 920 connected to cathodic bus 92 and 2) placing other electrodes 40 through appropriate inflection points of the nodes 910 connected to the anodic bus 91, a uniform dispersion of bipolar electrodes may readily be achieved. As there may be mechanical advantages to vary the spacing of nodes, other embodiments (not specifically illustrated here) readily may be designed to accomplish such advantages. In general, the large variety of possible active electrode spatial configurations as embodiments should be apparent.

When applied to the skin of a patient, the device strains and conforms to the skin. A number of temporary changes occur proportionately to the degree to which the device is in tension: the device elongates and necks down in cross-section; the strain on the material increases the pressure on the liquid or viscous material which is encapsulated; and contours of the welded area distort. Each of these temporary conditions facilitates measurement of the degree of movement/extension of a limb. In the case of the reduction in cross-section, a conductive medium with conductivity in the 12-15,000 microSiemens range has been observed to show a temporary, recoverable increase in resistance during elongation which is proportional to the elongation. The observation was consistent up to 225% strain. The temporary effect is driven by the geometric cross-sectional changes within the conductive region while in tension. Careful selection of the Poisson's ratio in the encapsulating films and the conductive media will enable tailoring the stress to the skin and the monitoring of the desired elongation range. The hydrostatic pressure increase within the encapsulated channel can be used with thin film pressure release valves or check valves, known to one in the art of fluid circuits. The permanent re-distribution of encapsulant into a small reservoir on the far side of a pressure cracking valve indicating a specific pressure would create a visual record of the distortion to the device and the limb beneath it. When applied across a joint in extension, the changes due to elongation of the device when the joint moves to a flexion position will correlate to a measure of range of motion.

In the lab, a maximum strain of products such as Kinesio® Tape have been observed to be 200%. U.S. Pat. No. 5,861,348 describes this product as herein incorporated by reference in its entirety. Embodiments of our device, capable of electrical connectivity at 200+% strain become viable tools in skeletal-muscular treatments. When applied across a joint in extension, the changes resulting from elongation of the device when the joint is in flexion will correlate to a measure of range of motion.

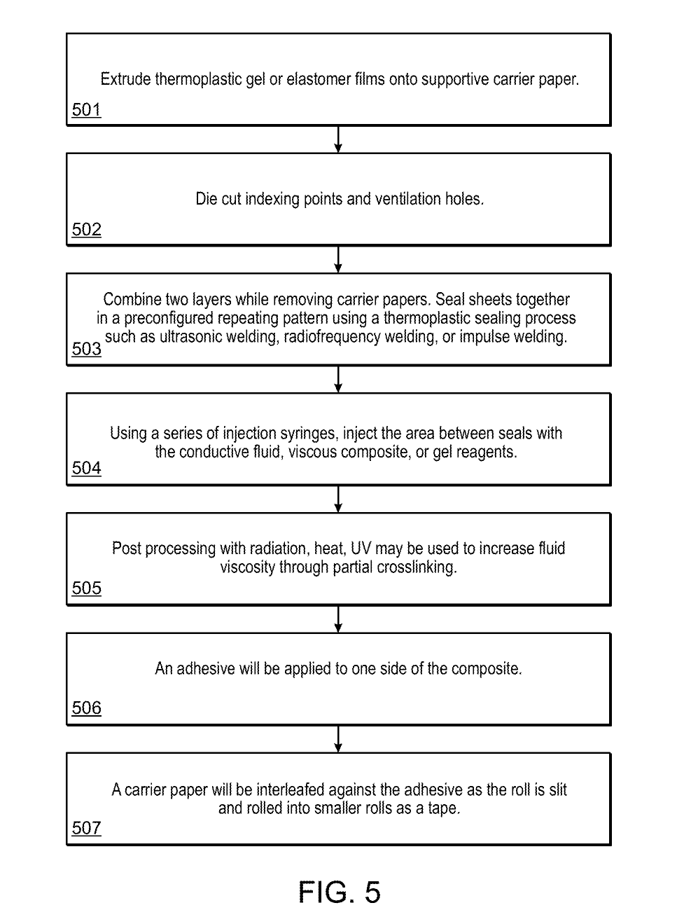

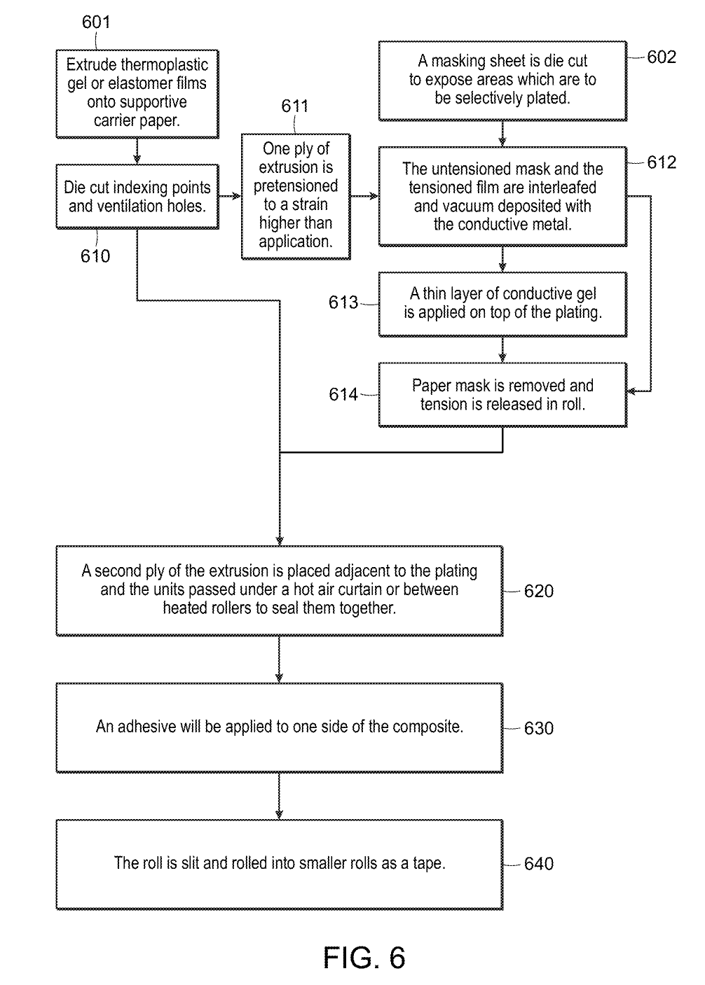

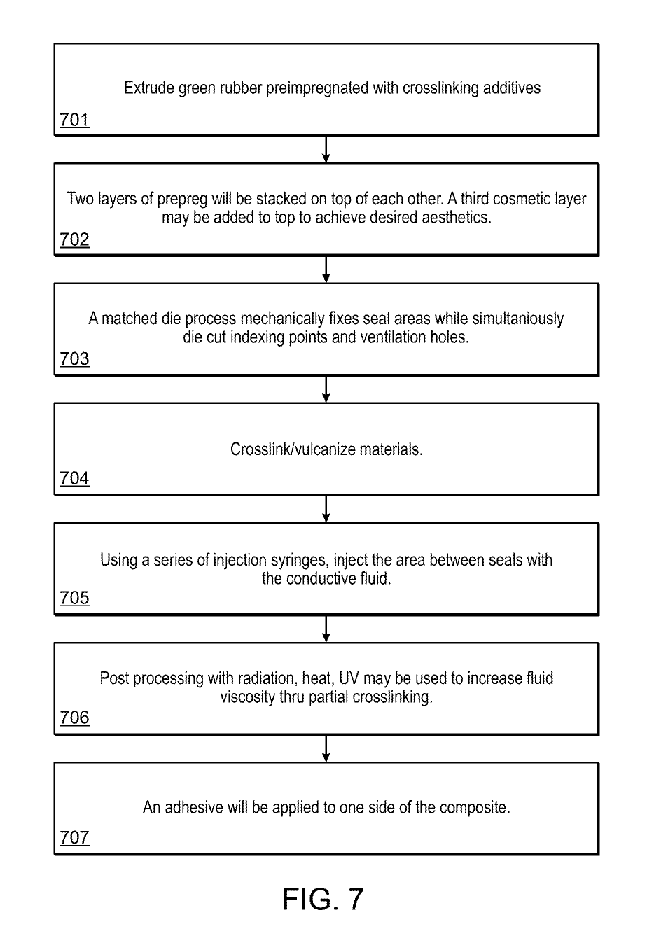

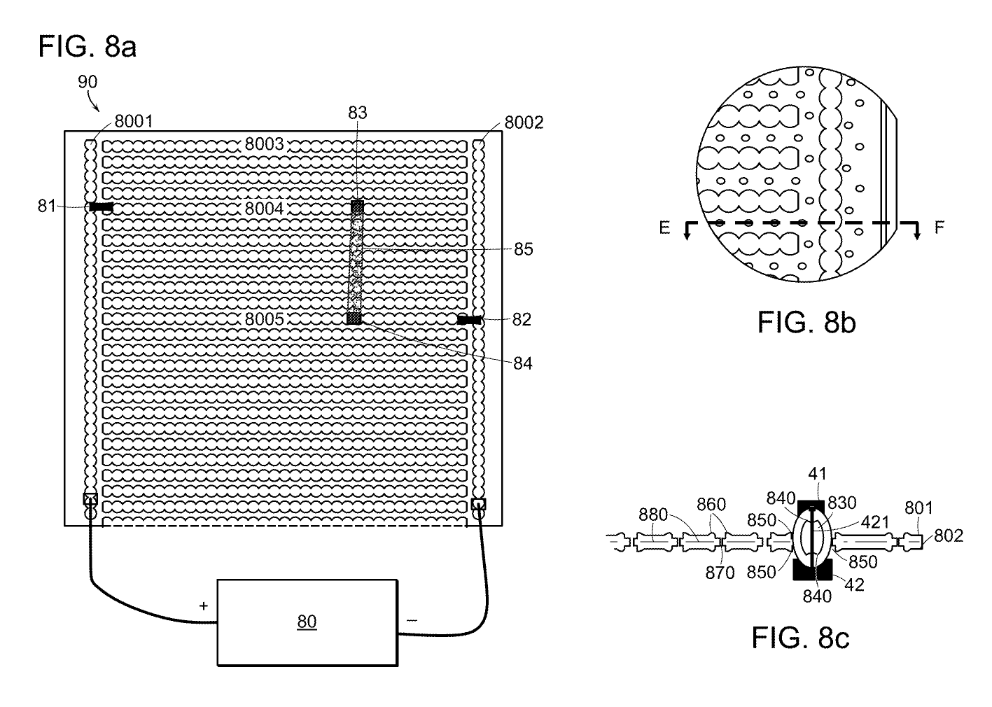

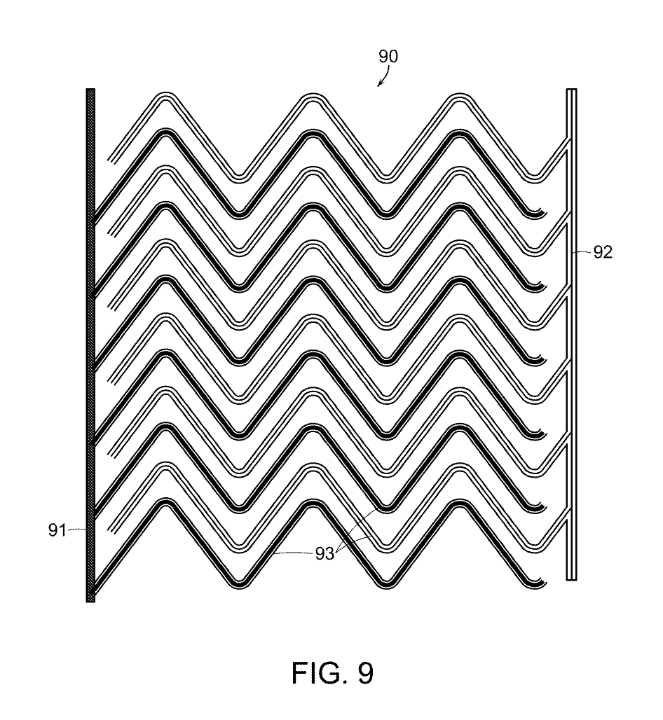

Refer to FIGS. 5, 6 and 7. FIG. 5 and FIG. 7 describe the processing of a tape 2000 utilizing a fluid or viscous conductive medium contained/encapsulated within an essentially insulative matrix. For the purposes of this disclosure, a viscous conductive medium is defined as exhibiting a viscosity in excess of 5000 centipoise. Fluid or fluid solutions are those having lower viscosity than that of viscous media. The use of the term gel denotes a subset of the broader class of viscous media. FIG. 6 describes processing of tape 2000 utilizing conductive plating that is selectively deposited upon the matrix material. Another processing option (no figure) for the placement of conductive medium within the matrix may involve use of metal foil that is sandwiched between the insulative matrix plies. To manufacture multimodal therapeutic device 100 having a composite tape 2000 as its platform, first (501, 601, 701) extrude gel (viscous medium) or elastomer film or green rubber onto supportive material (likely, carrier paper) The elastomer may be a thermoplastic elastomer or elastomer alloy, a vulcanizing rubber such as isoprene or neoprene, or a silicone. Examples of thermoplastic materials include polyurethane, styrene-butadiene alloys, polyolefin blends, Santaprene®, Dynaflex®, Versaflex®, Kraton®, Grilamid®. In the case of thermoset rubber, an optional step 702 may include the inclusion of a compatible cosmetic ply on the top surface as many rubber compounds are limited in colorability and decorative aptitudes. Next (502, 602, 703), using methods known in the art, through holes are die cut into the plies for a) indexing points (for alignment of plies) and b) ventilation holes (for removal of any processing by-products and/or for facilitating the removal of sweat (in operation) which could cause patient discomfort and possible deleterious electrical issues. Particularly, FIG. 7 requires a matched die pair for operation 703 as the additional goal of mechanically commingling the two green thermoset layers is necessary. Step 704 immediate follows step 703 and allows for the crosslinking of the thermoset materials prior to the insertion of liquid. For the process embodiments of both FIG. 5 and FIG. 7, the seal geometry and material features will play a significant role in the manufacture and function of the gel encapsulated approach. Refer to the further detailed description (in association with FIGS. 8a-8c) following the present manufacturing methods disclosure. Step 503 is to first remove supportive material/carrier paper from each of the two layers while laying up the matrix plies. The layed-up plies are then sealed using processes such as ultrasonic welding, radio-frequency welding, impulse welding with a resistive heating element, or solvent bonding. Next (504 and 705) a conductive medium is injected, or otherwise forced into the interior (between the seals) of a precursor of tape/platform 2000. The conductive viscous medium/fluid may consist of a suspension of polymeric thickener, metal or metalized particles, solvent, and ions. Thickeners may, for example, be a hydrogel, hydrosol, or hydrocolloid such that time stabilization of the conductive media is achieved. Materials which could be used include poly acrylic acids, polyvinyl alcohol, poly-N-vinylpyrrolidone, silicone, polyurethane, and polyamides and combinations thereof. The conductive elements will be dispersed, suspended, or held within a thickening network. A portion of the conductive media may be powders of known metal conductors (Ag, Cu, W, Sn, Mo, Ni) or may be spheres, rods, or nanostructures that are coated with conductive metals. There may also be an ionic conductor (such as silver chloride ions, styrene maleic anhydride, potassium chloride, or other halogen-based salt) working in combination with the thickener by circulating between colloid particles, or through an open polymer network. In some specific platform/tape embodiments for which the circulation of the conductive fluid is desired, a thickening of the conductive media may be desirable. Thickening has also improved shelf-life of the conductive dispersion and achieved desired rheological behavior. Steps 505 (and 706) allow for the thickening of the conductive media with the application of UV, heat, radiation, or other means of achieving activation energy in the reagents. An encapsulated construction should feature hermetically sealed wells to house the conductive gel/fluid. FIGS. 8a and 8c, illustrate an exemplary embodiment of discontinuous conducting layer/platform 90 having nodes 8003-8005 arranged essentially orthogonal to the alignment of and selectably connectable to the buses 8001 and 8002. FIG. 8b shows a magnification of platform 90 proximate to bus 8002 depicting ventilation holes 870 as well as regions 880 that are devoid of nodes, buses or ventilation or indexing holes. FIG. 8c is a cross-section through line EF illustrating top and bottom plies 801 and 802 bonded together. Weld region 850 is configured as a full perimeter weld around an open well between the two insulative films/plies 801 and 802. The weld creates discrete wells (e.g. 8001-8005 that will house the conductive gel/viscous material capable of serving as node and bus portions of discontinuous conducting platform 90. Appliances, such as an electrode (shown in FIG. 8c the electrode components first portion 41 and second portion 42 with through post there between), are shown inserted/punctured through a conductive channel 830. Given the alignment of line EF, an electrode (rather than a jumper) is shown as placed within a portion of (conductive channel 830) bus 8002 which is filled with conductive medium. Films 801 and 802 have material properties such that they demonstrate a strong needle seal behavior such as would be seen in subcutaneous injection ports and infusion sets. This resiliency and resistance to compression set enables the films to accommodate the conductive through post and to generate a functional seal under low fluid pressures. FIG. 8c shows regions 840 where the puncture deflects to accommodate the conductive post. This same needle-seal matrix property that enables manufacture will also enable subsequent configuration and reconfiguration of the active electrode/device array disposed within the platform 90. The needle seal properties exist throughout channels 830 and enable a large number of possible insertion/mounting positions for electrodes/appliances with no fluid leakage. Thus, unlike the fixed insertion sites 2010 shown in the embodiment of FIG. 2a-2c, a sealed well conductor presents little to no geometric limitations to electrode/appliance configurations. In addition to the welds made for sealing the electrical conductive material, seals 850 and ventilation holes 870, as shown, are provided through the device to prevent unwanted cultures next to the skin and within the device. Cavities in regions 880 (devoid of features) exemplify the independence of plies 801 and 802 distanced from welded points. Protrusions 860 show beads of matrix material which are generated as a side effect of the weld process. Finally, power source 80 is shown with its positive terminal in electrical communication with bus 8001 and with its negative terminal in electrical communication with bus 8002. Here, as in the previous embodiment depicted in FIG. 2c, nodes 8003 of conducting layer/platform 90 are not permanently in electrical communication with either bus 8001 or 8002. To illuminate how a pair of electrodes would be created and energized leading to, in this specific example, conventional whole muscle treatment (monopolar operation), jumper 81 provides electrical communication between node 8004 and bus 8001 while jumper 82 provides electrical communication between node 8005 and bus 8002. Therefore, node 8004 is of opposite polarity from node 8005. Region 85 simulates a region of living tissue which is to be stimulated by conventional monopolar means. The region between site/insertion point 83, located on node 8004 and insertion point 84, located on node 8005, approximates the boundaries of region 85. When electrodes are inserted into sites 83 and 84, platform 90 (embedded in device 100) is in position about region 85, and power source 80 is connected to buses 8001 and 8002, therapeutic stimulation may begin.

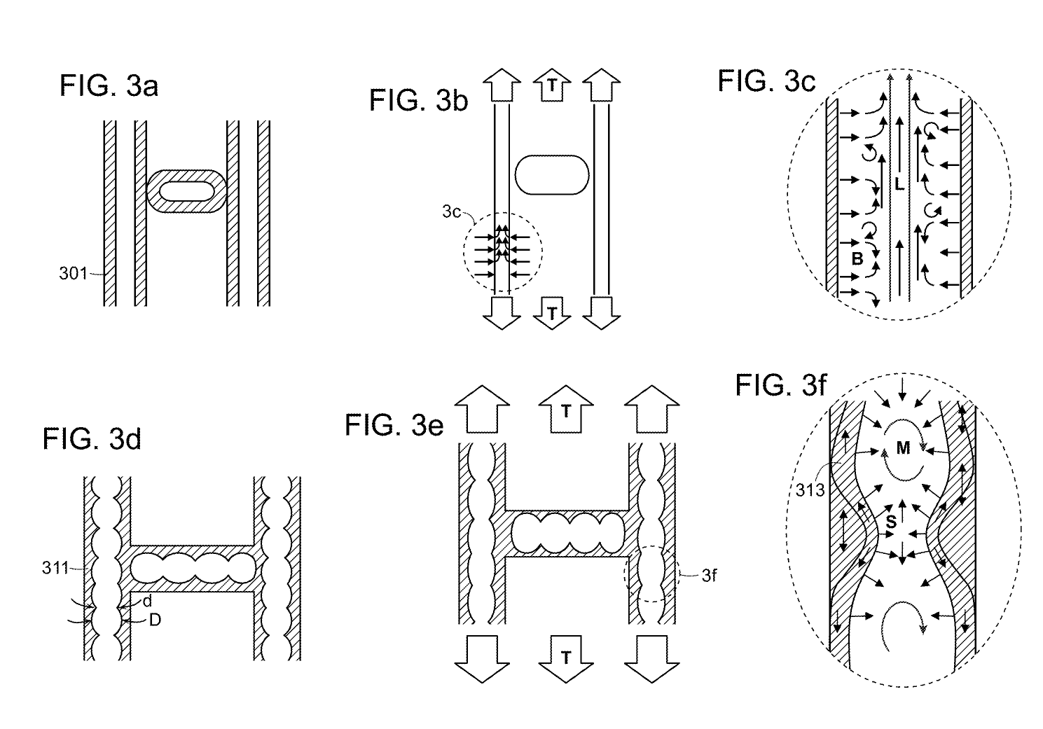

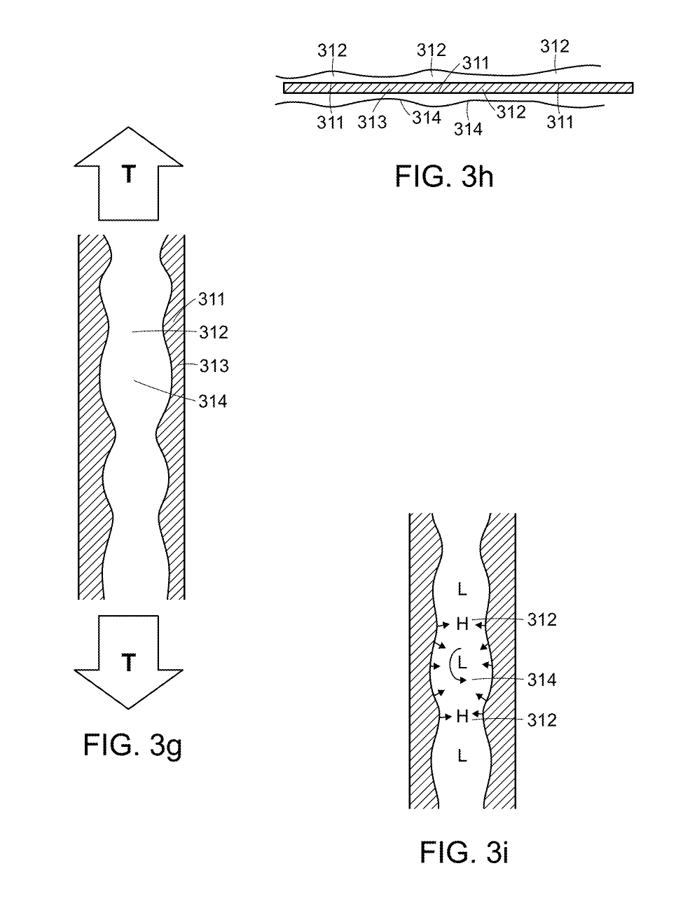

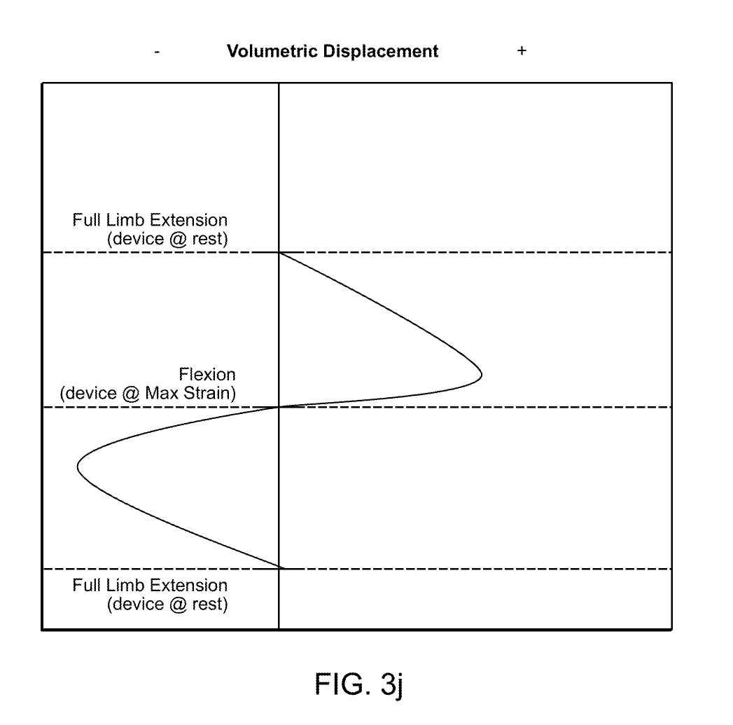

The encapsulated channels are designed to allow for circulation of the conductive gel/viscous medium. Such flow will occur during elongation and contraction during operation of device 100. For an example of how elongation will occur, consider a device 100 placed on the dorsal side of the leg. The platform will be forced to elongate in tension when the joint is in extension. The device will stretch longitudinally causing any longitudinally oriented channels to reduce in cross-sectional area. The narrowing side walls of these channels will force the viscous fluid conductive medium to flow. When the flexor muscle is stimulated and the joint is put in flexion, the dorsally mounted device will transition from its tension state to a relaxed state; the channel diameters enlarge when in the relaxed state creating a larger cross sectional area and a low pressure zone thereby reversing the direction of flow. Special features, geometric or other, may be designed into the platform affecting the thickness of the weld, the diameter of the channel, and the curvature of the weld profile which may serve to promote both flow and mixing during periods of mechanical loading of the platform. Variations in channel/well diameter, as shown to occur periodically in the channel embodiments of FIGS. 8a-8c, are exemplary of such features. FIGS. 3a-3c illustrate a straight walled sealed channel, the narrowing of the channel in tension, and the resultant flow profile within the channel, respectively. FIGS. 3d-3f parallel the previous illustrations applied to a channel of non-uniform (similar non-uniformity to that of the platform embodiment of FIG. 8a) cross-sectional area. The shaded regions shown by 301, 311, and 313 represent regions in which a first sheet/ply is welded to a second sheet/ply to encapsulate a conductive fluid. Unwelded regions will have two maintained plies which will not necessarily function/deform as a single structure. When elongated in tension (direction T), the straight-walled channel (FIGS. 3a-3c)will extend approximately 3 times the amount that it necks down due to tension (as would a typical Thermoplastic Elastomer (TPE) or Thermo Ether-ester Elastomer (TEEE) with a Poisson's ratio of 0.3). Further, the straight walled channel will have a typical fluid flow pattern featuring both a boundary area B where mixing occurs and a central area L of laminar flow. If, however, the channel is made of a periodically patterned feature which will change non-uniformly in cross-section due to stress concentrating features. (FIGS. 3d-3j illustrate such a construction), the varying channel cross-section (illustrated as a smaller d versus a larger D) will also reinforce non-uniform deformation in response to the hydrostatic pressure being applied to varying surface areas. The resulting distortion experienced by this irregular channel is expected to be non-uniform for the following reasons: 1) wall 311 depicts a thicker welded region than does 313, 2) when tension is applied the stress will be less in 311, due to increased localized thickness, therefore there will be less longitudinal and transverse elongation, 3) the thicker weld will also offer more localized resistance to hoop stress. The single ply sidewalls of region 313 will be more compliant and show the bulk of the deformation for this region of the matrix when the fluid pressure increases during tensioning. FIG. 3h depicts some thickness variations in the single ply encapsulant as fluid pressure seeks equilibrium. Region 314 has a larger surface area than 312. Region 313 will strain in accordance with the hoop stress placed on it by the fluid pressure. Because region 313 exhibits both a thin weld area and a larger open radius (compared with straight-wall), the single most significant volumetric expansion should happen in this region. Regional low pressure from this expansion should initiate turbulent flow of the conductive medium away from 312 toward/into 314. FIGS. 3f and 3i depict regions within the non-uniform channel of expected high pressure, H, and axial flow, S, and those with lower pressure, L, and turbulent mixing, M. Significant mixing will occur between regions which grow more and recover more rapidly in volume capacity and those which are more restrained. This mixing should be at its peak when the device in transitioning from full extension to its relaxed state or when it's changing from its relaxed state to its elongated state (graphically illustrated in FIG. 3j). Because the ionic portion of the conductor is dependent on the diffusion of ionic molecules to conduct, this mixing action will support the maintenance of superior electrical conductivity of the fluid in the mechanically dynamic system of the multimodal device 100.

For the processing alternative wherein the conductive section is formed by selective electroplating of a conductive layer, 611 is an advantageous step of pretensioning one of the extruded matrix plies. Die cutting of a mask (step 602) is performed to facilitate selective electroplating of the pretensioned matrix ply with conductive material. Next 612, the mask and the pretensioned ply are interleafed. Conductive metal is then deposited thereupon. A layer of viscous conductive material (perhaps, gel) is placed atop the conductive deposited coating 613. In step 614, the mask is removed and tension is released from the plated ply. The second extruded matrix ply is then (step 620) placed adjacent to the plated side of the plated ply and the assemblage is heated (or heated and compressed) to seal them together. An adhesive 630 may then be applied to a side of the sealed assemblage and the assemblage is cut 640 to form the desired tape.

Again refer to FIG. 1c and to FIGS. 1d-1h describing and illustrating use of exemplary embodiments of conductive layer 2001 along with associated configurations of activated insertion point/electrode sites 2010 for device embodiments 100 to afford multimodal stimulation both mechanically and electrically in the aforementioned dorsiflexion case study. Tensioned application of device 100 is shown on the left-most view of the right leg of the patient (FIG. 1c). In the center view, device 100 has a monopolar electrode configuration. Such a monopolar configuration is shown as having concentrated regions of one pole (say, cathode) electrodes placed on a first end of a muscle and another concentrated region of anode electrodes at another muscle end. This results in electronic signal being transmitted along the length of the muscle to be stimulated. Recall, for example, region 85 of FIG. 8a as an exemplary monopolar application.

Trigger points of muscles 101, 102, and 103 (denoted 1010, 1020, and 1030 respectively) are seen in the center and right-most views of FIG. 1c as well as in FIGS. 1d-1h. In the right-most view, device 100 has a bipolar electrode configuration. In this treatment scheme, aggregate bipolar electrode sites 1111, 1112, and 1113 are placed proximate muscle trigger points 1010, 1020, and 1030 of muscles 101, 102, and 103. The muscles are to be stimulated with signal focused proximate to the trigger points rather than with signal directed along the muscle body (monopolar). The aggregate bipolar electrode sites 1111, 1112, and 1113 are formed by creating essentially equal numbers of anodically and cathodically oriented through holes through which conductive material is to be passed providing electrical communication with the skin of the patient. The spacing between the individual bipolar electrodes will be selected based on the size of the patient, the amount of adipose tissue, and the required depth of penetration. One versed in the art of interferential stimulation would possess the knowledge of signal transmission and interaction in human tissues to ascertain the appropriate spacing.

FIGS. 1d, 1e, and 1f show details, with respect to electrode site selection, node layout, bus and hexagonal cell layout (FIG. 1e) of three monopolar device embodiments. FIGS. 1g-1h show similar details of device embodiments designed to provide bipolar stimulation. These five examples help to illustrate the extent to which the therapeutic devices disclosed herein are tailorable to a variety of clinical conditions and treatment schemes. In FIG. 1d, device 100 has buses 200 and 300 disposed proximate either edge aligned along a long axis. The first bus is anodic relative to the cathodic second bus when a power source 80 is in electrical communication with the buses. Due to real life geometrical considerations, bus 200 is only visible down the front of the leg while bus 300 is only visible wrapped about the top of the foot. In this embodiment, note that the nodes/pathways are essentially linear and disposed essentially orthogonal to the buses. Jumpers 20 are connected selectively to accomplish the completion of the circuit powered by the source. For example, in the monopolar configuration of FIG. 1d, anodic jumpers 20 are used to activate pathways 21 (six adjacent nodes with nineteen individual electrode sites creating an aggregate anode 120 for stimulating muscle 101) and 22 (four adjacent nodes with six individual electrode sites creating an aggregate anode 140 for stimulating muscle 102) completing the anodic circuit with anodic bus 200. In a similar way, cathodic jumpers 30 are used to activate pathways 33 (two adjacent nodes with five individual electrode sites creating an aggregate cathode 150 for stimulating muscle 103) completing the cathodic circuit with cathodic bus 300. In a similar manner, aggregate electrode sites 110, 130, and 160 are selectively formed using jumpers linked to the appropriate bus. When device 100 is powered with jumpers 20 and 30 secured, each of muscles 101, 102, and 103 will be stimulated via what was previously connoted whole muscle stimulation.

FIG. 1e illustrates another device embodiment utilizing hexagonal cells akin to those shown in FIGS. 2a-2c. An isolated group 2400 of contiguous hexagonal cells will be afforded a continuous conductive pathway utilizing, for example, a single jumper 20 connection to anodic bus 200 (and a single jumper 30 connection to cathodic bus 300. Therefore, in this configuration, as many individual electrode sites as are desired (within the contiguous group of cells) may be activated (by placing electrodes therein), each having the same polarity. As they will necessarily have the same polarity, this results in possible whole muscle stimulation with one end of the muscle having a first polarity and the second end having the opposite polarity (analogous to the embodiment of FIG. 1d.

FIGS. 1f and 1h illustrate yet other embodiments utilizing zigzag shaped nodes. The possibility for either monopolar or bipolar (stimulation focused upon the trigger point of a muscle is possible. The embodiment shown in FIG if is monopolar, with two sets of active electrode sites positioned at either end of a muscle. For example, with respect to muscle 101, eight anodic sites 40 are activated via electrical communication with two nodes 400. The nodes in this embodiment are permanently connected to a bus; adjacent nodes are connected to the opposite polarity bus. Cathodic sites are analogously created and shown to be located on an opposing muscle end. The embodiment of FIG. 1h is bipolar with sites of opposite polarity (formed from adjacent nodes connected to opposite polarity buses 91 and 92) located proximate trigger point 1010 (for muscle 101) etc.

FIG. 1g shows a further embodiment utilizing linear nodes orthogonal to the buses. Nodes proximate to muscle trigger points are jumpered alternately to the anodic and cathodic buses. In the figure, jumpers 20 are shown connecting nodes 2200 to anodic bus 200 while jumpers (not visible) connect nodes 2100 to cathodic bus 30. In this way, electrodes of opposite polarity are active in close proximity to each other and to the trigger point 1010 of muscle 101 (and the other two muscles and associated trigger points) to provide aggregated bipolar electrodes 1111 to accomplish target centered stimulation.

For some applications, embodiments of the device may incorporate a pharmaceutical material (not shown), such as, but not limited to: analgesic, anesthetic, anti-inflammatory, or vitamin to be dispensed by electromotive drug administration.

Although the invention has been described with reference to several embodiments, it will be understood by one of ordinary skill in the art that various modifications can be made without departing from the spirit and the scope of the invention, as set forth in the claims.

What is claimed is:

1. A therapeutic device for stimulation of living tissue, the stimulation may be mechanical, electrical, chemical, sensory and combinations thereof, the device comprising:
a matrix capable of attaching to living tissue, the matrix being sufficiently elastic so that when attached to the tissue, the matrix is capable of applying force upon the tissue and compliantly responding to force resultant from motion of the tissue; and
an electrically conductive region contained within the matrix, so that selectable locations of the device are capable of being in electrical communication with a power source and with the tissue.

2. The device of claim 1 wherein each selectable location further comprises:
an electrode in electrical communication with the tissue.

3. The device of claim 1 wherein the conductive region comprises a viscous conductive medium.

4. The device of claim 1 wherein the conductive region comprises a fluid solution.

5. The device of claim 1 wherein the conductive region comprises metal foil.

6. The device of claim 1 wherein the conductive region comprises electroplated metal.

7. The device of claim 1 where in electrical communication is maintained when the device has been strained to 225%.

8. The device of claim 1 capable of, in response to device elongation and contraction caused by tissue motion, facilitating measurement of motion of a limb, the limb proximal to the tissue.

9. The device of claim 1 capable of, in response to device elongation and contraction caused by tissue motion, facilitating measurement of an angle exhibited by a joint, the joint proximal to the tissue.

10. The device of claim 1 further comprising:
an outer adhesive layer bonded to the matrix.

11. The device of claim 1 further comprising:
a layer of pharmaceutical material capable of being delivered to the tissue via electromotive drug administration.

12. The device of claim 11 wherein the pharmaceutical material is selected from the group consisting of analgesic, anesthetic, anti-inflammatory, vitamin, and combinations thereof

13. The device of claim 3 wherein flow of the medium is promoted in response to force exerted upon the matrix.

14. The device of claim 4 wherein flow of the solution is promoted in response to force exerted upon the matrix.

15. A method for stimulating living tissue of a subject, the method comprising:
selecting stress, electrical triggering and sensory subject locations;
providing the therapeutic device of claim 1;
mapping the device by spatially matching the subject locations with the selectable locations of the device;
attaching the mapped device to the subject; and
powering the device.

16. A method of making the device of claim 6 comprising:
extruding thermoplastic material on carrier material to make first and second plies;
creating a mask;
applying tension to the second ply;
affixing the mask to the tensioned second ply;
depositing conductive material on masked second ply;
applying viscous conductive medium upon deposited conductive material;
removing mask from the second ply;
releasing tension from the second ply;
laying up the first ply with the second ply; and
sealing the plies together.

17. A method of making the device of claim 3 comprising:
extruding thermoplastic material on carrier material to make first and second plies;
sealing the first to the second ply leaving predetermined wells between the plies; and
injecting the viscous conductive medium into the wells.

18. The method of claim 17 further comprising:
providing adequate activation energy to crosslink the plies.

19. A method of making the device of claim 4 comprising:
extruding thermoplastic material on carrier material to make first and second plies;
sealing the first to the second ply leaving predetermined wells between the plies; and
injecting the fluid solution into the wells.

20. The method of claim 19 further comprising:
providing adequate activation energy to crosslink the plies.
